(19) United States
(12) Patent Application Publication
(10) Pub. No.: US 2016/0194719 A1
(43) Pub. Date: Jul. 7, 2016

(54) A BIOMARKER OF BREAST CANCER

(71) Applicant: NATIONAL UNIVERSITY OF IRELAND, GALWAY, Galway (IE)

(72) Inventors: Peadar Waters, Waters (IE); Michael Kerin, Galway (IE); Roisin Dwyer, Galway (IE)

(21) Appl. No.: 14/893,631

(22) PCT Filed: May 27, 2014
(86) PCT No.: PCT/EP2014/060991

§ 371 Date: Nov. 24, 2015

(30) Foreign Application Priority Data
May 27, 2013 (GB) 1309462.8

(51) Int. Cl. C12Q 1/68

(52) U.S. Cl. CPC C12Q 1/6886; C12Q 2600/158; C12Q 2600/178; C12Q 2600/136; C12Q 2600/112; C12Q 2600/118

(57) ABSTRACT

The invention provides methods of and diagnostic kits for the detection of breast cancer or to assist in assessing the prognosis, cancer comprising a reagent capable of identifying a circulating miRNA, the miRNA being the miR-138 biomarker. The invention also provides methods of identifying a therapeutic agent capable of preventing or treating cancers, including breast cancer, comprising testing the ability of the potential therapeutic agent to reduce the expression of miR-138.

### FIELD OF THE INVENTION

The present invention relates to the diagnosis of breast cancers. The goal of the invention is to allow early detection and diagnosis of breast cancers by providing a minimally invasive test which is both sensitive and specific. The invention provides a marker for breast cancer. The marker can be used to differentiate breast cancer from benign breast disease. It can also be used to differentiate between different tumour subtypes and different stages of disease. It can further be used to identify patients with basal breast cancers. It may also find use in determining the prognosis of a breast cancer patient and prediction of a response to cancer treatment.

### BACKGROUND TO THE INVENTION

Breast cancer is an extremely important disease in Irish Society. It is the most commonly diagnosed malignancy in Irish women (with over 2,600 new diagnoses annually), and accounts for the greatest number of cancer related deaths in women. With 1 in 8 Irish women being diagnosed with breast cancer, it is a disease which impacts, in some way, most people's lives. Breast cancer is an extremely prevalent disease accounting for the greatest number of cancers diagnosed and the greatest number of deaths from cancer in women in Ireland. When diagnosed and treated early, breast cancer is a highly curable disease, and the past decade has witnessed major advances in its management. However, many women continue to die from this disease process as a consequence of late diagnosis, with incurable metastases at the time of presentation. The focus of the invention is on the development of a sensitive, specific, minimally invasive biomarker for breast cancer that can be utilised to detect tumours, as well as being applied to monitoring the response to treatment, and detecting disease recurrence. Most studies divide breast cancer into four major molecular subtypes:
Luminal A
Luminal B
Triple negative/basal-like
HER2 type

Basal-like tumours account for about 11% of all breast cancers. These tumours have cells with features similar to those of the outer (basal) cells lining the mammary ducts. Basal-like tumours tend to express HER1 and/or cytokeratin 5/6 proteins and most contain p53 mutations. Most triple negative tumours are basal-like and most basal-like tumours are triple negative. However, the two are not synonymous. About 15 to 20 percent of breast cancers are triple negative or basal-like. These tumours tend to occur more often in younger women and African American women. Most BRCA1 breast cancers are both triple negative and basal-like.

Triple negative/basal-like tumours are often aggressive and have a poorer prognosis (at least within the first five years after diagnosis) compared to the estrogen receptor-positive subtypes (luminal A and luminal B tumours). Triple negative/basal-like tumours are usually treated with some combination of surgery, radiation therapy and chemotherapy. These tumours cannot be treated with hormone therapies or trastuzumab (Herceptin) because they are hormone receptor-negative and HER2/neu-negative. The genes linked to basal-like tumours are not well understood at this time and thus, targeted therapies do not yet exist. However, potential targets for future therapies include the EGF receptor, aB-crystallin and cyclin E.

The focus of the invention is on the development of sensitive, specific, minimally invasive biomarkers for breast cancer, that can be utilised to detect early tumours, and detect disease recurrence as well as differentiating between different tumour subtypes and different stages of disease.

MiRNAs are a class of small, non-coding RNA fragments that have captured the attention and innovation of the scientific community since their discovery almost twenty years ago. The discovery that MiRNAs are dysregulated in several disease processes, including carcinogenesis, has unveiled their putative role as disease-specific biomarkers and therapeutic targets. Little, however, it is known about the relationship between circulating levels of miRNAs and the characteristics of specific cancers.

Currently diagnosis of breast cancer involves a combination of clinical examination, radiological imaging and an invasive tissue biopsy, to provide histological confirmation. Mammography is currently considered the gold standard for diagnosis, yet it is not without its constraints, with both ionisation exposure and a false positive rate of up to 10%. In fact, screening mammography is reported to have a sensitivity ranging from only 62.9% to 87%. Small curable early cancers can potentially be missed. When diagnosed promptly, women are more likely to have early stage disease, confined to the breast. These cases are amenable to breast conserving surgery. In cases where tumour cells have metastasized to the lymph nodes at the time of diagnosis more extensive local surgery and auxiliary surgery (an auxiliary lymph node clearance is often required). This procedure carries the potential for severe complications, including lymphoedema, which impacts extensively on one's quality of life. More over, tumour cells in the lymph nodes may be an indicator of distant metastases elsewhere, that are undetectable by current strategies. Current practice demands that the majority of these women receive adjunct chemotherapy and/or hormonal therapies. The decision to commence such regimens should not be taken lightly as each of these treatment modalities has an associate cluster of adverse effects. The appropriateness of adjunct therapy is usually based on a culmination of histological and patient factors. Only two predicted markers are validated and routinely assessed in the management of breast cancer. One of these is Oestrogen Receptor (ER) status and the other is Her2/neureceptor status. These markers indicate that there is a likely benefit to be achieved from treatment with hormonal therapies or Trastuzumab, respectively.

MicroRNAs are a class of small non-coding RNA fragments that are ideal biomarker candidates and therapeutic targets. MiRNAs have been demonstrated to play a key role in practically all aspects of the cell cycle. They function at a post-transcriptional level to cause either transcriptional cleavage or transcriptional repression. MiRNAs are aberently expressed in almost all pathological conditions, including carcinogenesis and have a putative role as oncogenes or tumour suppressor genes. MiRNAs have the advantage that they can be detected in the systemic circulation, and thus diagnostic assay is based on miRNAs are not invasive.

The concept of breast screening is now accepted internationally and is associated with an improved outcome from breast cancer due to its earlier diagnosis. However, it is not without its drawbacks; a high proportion of lobular carcinomas are mammographically occult dense breasts make the interpretation of the mammogram difficult and there are high false negative rates. Therefore, the development of a blood based diagnostic tool would be a significant advantage as it would permit screening to begin at a younger age and could potentially successfully diagnose tumours which are not detectable by mammography.

Lee et at (PLOS ONE, vol 7, Issue 12, December 2012) describe NGAL (neutrophil gelatinase-associated lipocalin) expression being associated with tumourigenesis and the phenomenon being eliminated by overexpression of miR-138, and NGAL antibody. Thus miR-138 regulates NGAL expression. The researchers did not study native levels of miR-138.

### OBJECT OF THE INVENTION

It is an objective of the present invention to identify novel biomarkers which could be used in the diagnosis and/or prognosis of breast cancer. It is a further objective to identify a minimally invasive marker for use as an adjunct in breast cancer diagnosis and/or prognosis. A still further object is to provide a test, which is both sensitive and specific. Yet another object is to provide a blood test for the diagnosis, or the assessment of prognosis of breast cancer.

### SUMMARY OF THE INVENTION

According to the present invention there is provided a diagnostic kit for the detection of breast cancer or to assist an assessment of prognosis, comprising reagents capable of identifying at least a portion of a circulating miR-138 biomarker.

The reagent may be an oligonucleotide probe capable of binding to at least a portion of a circulating miR-138 biomarker, or it may be an antibody, labelled or unlabelled, directed against the miR-138 biomarker. The label may be radioactive, colorimetric, or enzymatic.

The portion of the miR-138 marker may be the nucleotide sequence AGCUGGUGUUGUGAAUCAGGCCG. This sequence is located on the chromosome Chr3:44155726-44155748.

The kit may be adapted for performance of an assay selected from a real-time PCR assay, a micro-array assay, histochemistry assay or an immunological assay.

The kit may further comprise reagents or vessels/receptacles for the collection or stabilisation of whole blood or blood derived materials.

The kit is particularly suited for use in differentiating between different tumour subtypes and/or different stages of disease, as well as in basal breast cancer detection or assessment.

The invention also provides a method of identifying a therapeutic agent capable of preventing or treating cancers, including breast cancer, comprising testing the ability of the potential therapeutic agent to enhance the expression of at least one circulating miRNA selected from the group comprising miR-138, the enhancement being in comparison with the expression level before treatment with the therapeutic agent.

In a still further aspect the invention provides for use of a circulating miR-138 to detect breast cancer, or to stratify patients according to expected prognosis. Breast cancer is detected, or patients stratified if the level of circulating miR-138 is higher than that in a normal control. The normal control may be a healthy individual, or it may be a patient with a better or worse prognosis or greater or lesser degree of breast cancer.

In this use the detection may be carried out in a blood sample or a sample derived from blood.

The invention also provides a method of detecting or screening for early stage breast cancers comprising analysing a sample of blood taken from a patient for the presence of the biomarker miR-138, the presence of the miRNA in the sample indicating the presence of breast cancer. The presence of miR-138 in the sample may be relative to a normal healthy control or another patient or group of patients with breast cancer or a particular type of breast cancer.

### BRIEF DESCRIPTION OF THE DRAWINGS

FIG. 1—Circulating miR-138 levels in breast cancer patients and healthy controls.

FIG. 2—Tissue miR-138 levels in normal, benign and malignant breast samples. MiR-138 levels were found to be significantly altered across tumour epithelial subtypes (p<0.01).

FIG. 3—Tissue miR-138 levels were found to be significantly altered across disease stages (p<0.005).

### DETAILED DESCRIPTION OF THE INVENTION

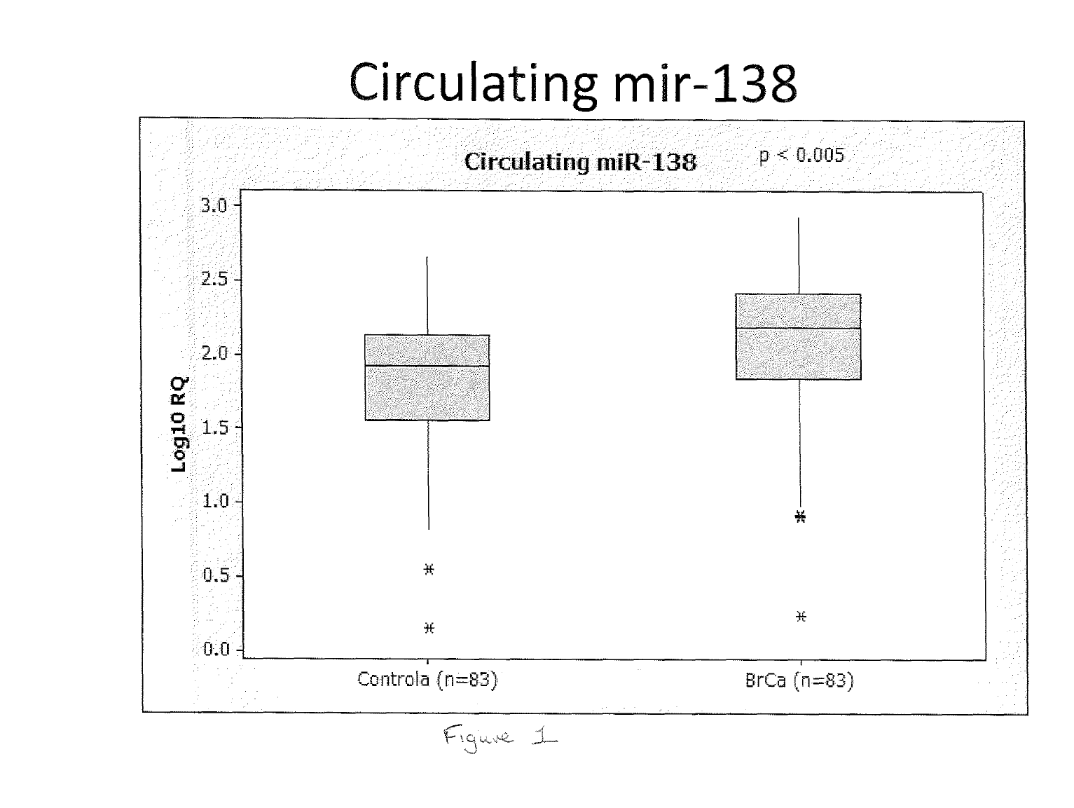

Serial samples of blood were harvested from tumour-bearing mice 1, 3 and 6 weeks following tumour induction, and tumour volume was monitored weekly. Whole blood samples were stored at 4° C. and tissue samples harvested at week 6 study termination were stored at −80° C. miRNA was extracted from all murine blood and tissues samples for analysis of microRNAs that were significantly altered between week 1 and week 6 of tumour development. The data was then analysed and the targets ranked based on the most significant change observed between week 1 and 6, with a requirement for the same target to be dysregulated in at least four animals. The top ranked microRNAs (including miR-138) were then analysed and validated across all murine samples (n=60) by RQ-PCR. Mir-138 was shown to be upregulated during disease progression, and further analysis of all murine samples by RQ-PCR demonstrated significantly higher levels of Mir-138 in animals with a high tumour burden compared to those with minimal disease. Patient samples were then selected to determine whether miR-138 was also elevated in breast cancer patients compared with healthy controls. Circulating miR-138 was measured in n=83 breast cancer patients and n=83 healthy controls with no history of breast cancer (FIG. 1). Mir-138 was found to be significantly elevated in patients with breast cancer (Mean [symbol] SEM; 2.05 [symbol] 0.06 Log Relative Quantity (RQ)) compared to healthy controls (1.83 [symbol] 0.05, p<0.005, FIG. 1).

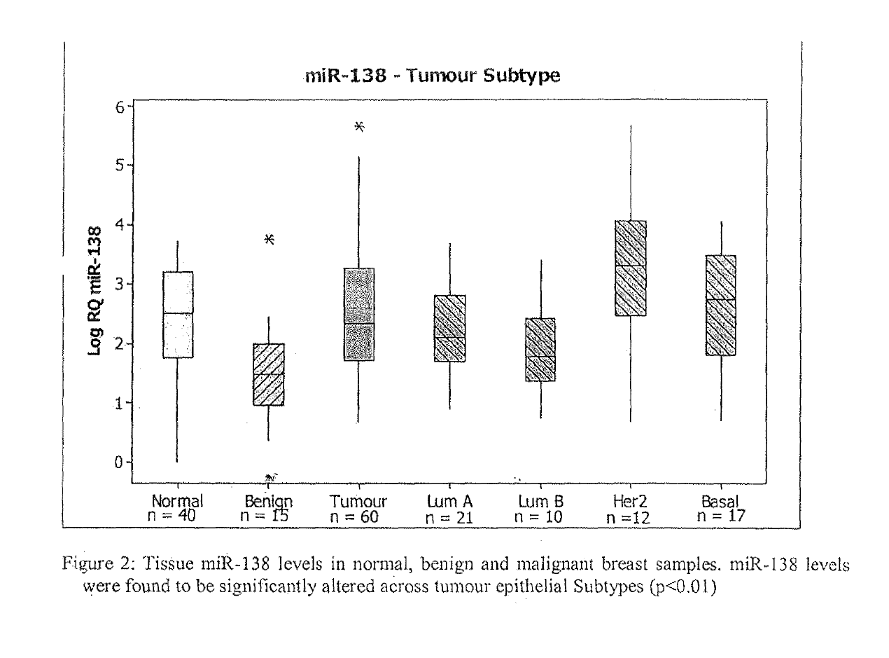

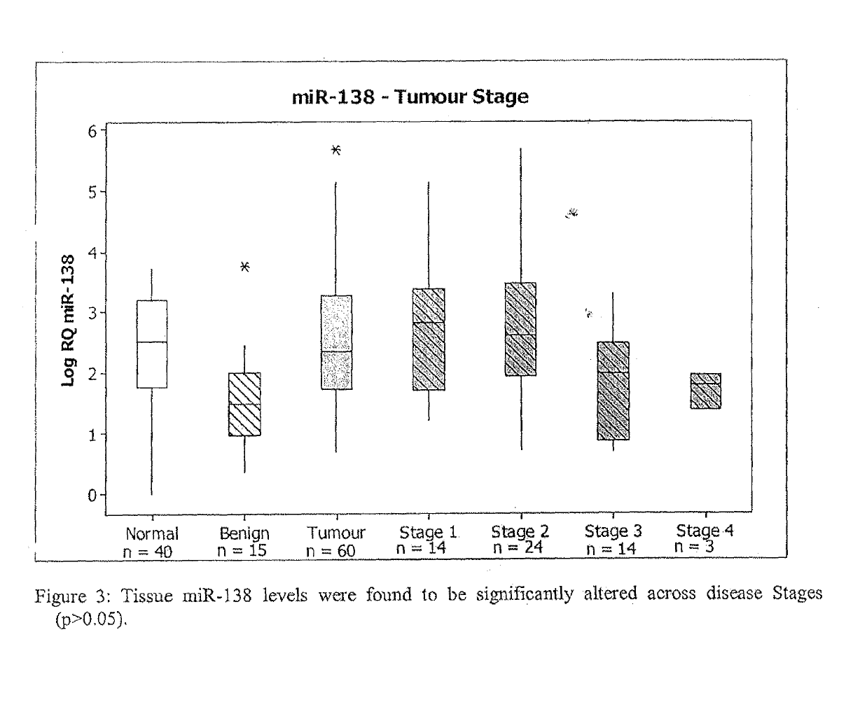

The level of MiR-138 was also determined by RQ-PCR in patient tissue samples including breast cancer (n=50), benign breast disease (n=15) and normal healthy breast tissue (n=40). miR-138 was found to be significantly elevated in breast cancer compared to benign breast disease (p<0.01, FIG. 2). Further within the breast cancer cohort, tissue miR-138 levels were found to be significantly altered across different tumour Subtypes (p<0.01, FIG. 2) and disease Stage (p<0.01, FIG. 3).

### Example

To conduct the assay, total RNA is extracted from a blood sample. The RNA is then reverse transcribed and then subjected to a Singleplex TaqMan™ microRNA assay reaction using a TaqMan universal PCR mix. Real time PCR amplification is then conducted using an Applied Biosystems Real-Time PCR system and the data is then analysed.

MicroRNA was extracted from 50 μl of whole blood using an amended version of the TRI Reagent® BD technique (Molecular Research Center, Inc., Cincinnati, Ohio), as previously described [1]. Collected RNA was stored at −80° C. The miRNA concentration and purity were assessed by NanoDrop™ 1000 spectrophotometry (Nanodrop Technologies, Wilmington, Del., USA) and Agilent Bioanalyser (Agilent technologies, Germany).

100 ng of mature microRNA was reverse transcribed using the MultiScribe™-based High-Capacity cDNA Archive Kit (dNTP 100 mM, RT Buffer 10×, RNase Inhibitor 20 U/μl, Stem loop primer 50 nM, MultiScribe RT 50 U/μl) (Applied Biosystems). The resulting cDNA was analysed by ABI 79000 Fast real-time PCR system (Applied Biosystems). PCR reactions were carried out in final volumes of 10 μl using a 7900 HT Fast Real-Time PCR System (Applied Biosystems). Briefly, reactions consisted of 0.7 μl cDNA, 1× TaqMan® Universal PCR Master Mix, 0.2 μM TaqMan® primer-probe mix (Applied Biosystems). The RQ-PCR cycle comprised of, 10-minute incubation at 95° C. followed by a 40 cycles at 95° C. for 15 seconds and 60° C. for 60 seconds. The use of an Inter-assay control on each reaction allowed comparison of data across plates, and all reactions were carried out in triplicate with a standard deviation of <0.3 considered acceptable. miRNA-16 was used as an endogenous control to standardize miRNA expression [2]. The relative quantity of miRNA expression was calculated using the comparative cycle threshold (ΔΔCt) method [3].

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### REFERENCES

1. Heneghan H M, Miller N, Kerin M J (2010) Systemic microRNAs: novel biomarkers for colorectal and other cancers? Gut 59: 1002-1004; author reply 1004.
2. Davoren P A, McNeill R E, Lowery A J, Kerin M J, Miller N (2008) Identification of suitable endogenous control genes for microRNA gene expression analysis in human breast cancer. BMC Mol Biol 9: 76.
3. Livak K J, Schmittgen T D (2001) Analysis of relative gene expression data using real-time quantitative PCR and the 2(-Delta Delta C(T)) Method. Methods 25: 402-408.

1. A diagnostic kit for the detection of breast cancer or to assist in assessing prognosis of breast cancer disease or in stratifying patients with breast cancer, comprising a reagent capable of identifying at least a portion of a circulating miRNA, the miRNA being the miR-138 biomarker.

2. A kit as claimed in claim 1 wherein the reagent is an oligonucleotide probe capable of binding to at least a portion of a circulating miR-138 biomarker, or an antibody directed against the miR-138 biomarker.

3. A kit as claimed in claim 1 or 2 wherein the portion of the miR-138 marker is the nucleotide sequence AGCUGGUGUUGUGAAUCAGGCCG.

4. A kit as claimed in any preceding claim adapted for performance of an assay selected from a real-time PCR assay, a micro-array assay, histochemical assay or an immunological assay.

5. A kit as claimed in any preceding claim for use in detecting basal breast cancer.

6. A kit as claimed in any of claims 1 to 4 for use in differentiating between different tumour subtypes.

7. A kit as claimed in any of claims 1 to 4 for use in differentiating between different stages of breast cancer disease.

8. A method of identifying a therapeutic agent capable of preventing or treating cancers, including breast cancer, comprising testing the ability of the potential therapeutic agent to alter the expression of the circulating or tissue levels of miRNA miR-138.

9. Use of the circulating miRNA R-138 to detect breast cancer, or to stratify patients according to expected prognosis, or to assess the efficacy of a medical treatment.

10. Use as claimed in claim 9, wherein the detection is carried out in a blood sample or a sample derived from blood.

11. A method of detecting or screening for breast cancers comprising analysing a sample of blood taken from a patient for the presence of the biomarker miR138, the presence of the miRNA in the sample indicating the presence of breast cancer in the patient.


SEQUENCE LISTING (1 sequence)

SEQ ID NO: 1 - RNA, 23 nt, Artificial Sequence
  Synthetic sequence - portion of miR-138 biomarker
agcugguguugugaaucaggccg